(19) United States
(12) Patent Application Publication
(10) Pub. No.: US 2025/0151669 A1
(43) Pub. Date: May 15, 2025

(54) APPARATUS FOR GROWING CONSUMABLE PRODUCTS

(71) Applicant: Matan SHABAT, Eilat (IL)

(72) Inventor: Matan SHABAT, Eilat (IL)

(21) Appl. No.: 18/838,628

(22) PCT Filed: Feb. 16, 2023
(86) PCT No.: PCT/IL2023/050172

§ 371 Date: Aug. 15, 2024

Related U.S. Application Data
(60) Provisional application No. 63/310,830, filed on Feb. 16, 2022.

(51) Int. Cl. A01G 9/16; A01G 31/02; C12M 1/00

(52) U.S. Cl. CPC A01G 9/16; A01G 31/02; C12M 23/52

(57) ABSTRACT

A system and apparatus providing a mobile module for the growing and delivering agricultural products and in particular hydroponically or/and cultured products under controllable environments. The apparatus having two housings including at least one primary housing having a controllable cultivating environment and a secondary housing.

### FIELD OF THE INVENTION

The present invention generally relates to growing products, and in particular, to a system and apparatus for the controlled growing and delivery of hydroponically or/and cultured products within an apparatus that is functional in varying environmental conditions.

### BACKGROUND OF THE INVENTION

Agricultural crops are traditionally grown in soil using traditional planting, growing and harvesting methods. Growing crops in this traditional manner requires a vast investment and corresponding risk. To be successful traditional agriculture generally require large area farming land, large volumes of water and fertilizer, specialized plowing machinery, seeding and harvesting vehicles, and hours of manual labor so as to increase the likelihood of producing significant crop yields. Unfortunately, the crop yield success rate is largely not controllable and related to uncontrollable factors such as weather conditions, environmental factors beyond the control of the farmer. Moreover, traditional agricultural practices are further limited due to crop related limitations such as specific growing seasons and geographic locations. Accordingly, it is generally understood that plants require a correct combinational environment of light, temperature, oxygen, nutrients, and water, etc., that are combined with attention and care required to allow the plant to continue to grow for a certain time period or life.

In an attempt to improve on traditional agricultural practices recently hydroponic growing has been developed. Hydroponic growing techniques are used to grow crops in a controlled environment to avoid the aforementioned problems. Generally, hydroponic techniques involve the growing of vegetation without soil by submersing seeds and/or plant roots in a mineral nutrient-enriched water solution or by spraying the seeds and/or plant roots with such a solution. The seeds/roots absorb nutrients from the solution and grow without the need for soil. While existing hydroponic growth techniques have addressed some of the problems with traditional soil-based agriculture, however both techniques are problematic when considering supply chain and/or delivery of the crop yield, and the time to market and/or consumer of the agricultural good.

In traditional hydroponic or aquaponic cultivating systems, a plant's roots, or section of the plant's roots, are not directly supported by a solid medium. The invention allows plant cultivation wherein roots are suspended within an aqueous solution and are supported by a medium permitting downward and lateral growth. The invention root trellis structure results in a healthy branched root system, greater plant biomass, and an overall increase in plant yields than prior art hydroponic systems.

When grown hydroponically, plants will generally flourish in a manner superior growth in normal soil medium. This is, in part, because there is a greater availability of nutrients when supplied to the root with a hydroponic solution as opposed to a normal soil medium. Hydroponically grown plants begin growth quickly and grow faster than they would in a soil medium and fruits ripen earlier. Additionally, with hydroponic gardening, plants require less space in which to grow due to the fact that access to nutrients is not constrained, thereby permitting the plants to feed and breathe much more easily than they could in soil. However, benefits of the soil are typically lost when utilizing hydroponics, resulting in a less branched root systems, an underdeveloped root system.

Frequently, typical support devices for plant growth are garden trellises and often made from interwoven pieces of wood, bamboo, metal or plastic, are coming to provide support to climbing plants are vines. Trellises and wire cages are conventionally employed to support the weight of the plant during growth and especially before harvest.

Conventional food supply chains often include a source or initial supplier of raw ingredients for food products for human consumption, such as plant-based or animal-based ingredients. The ingredients are often transported from the source to one or more processing facilities, where the raw ingredients are prepared into food products including one or more intermediate ingredients and eventually prepared into marketable food products intended for direct human consumption. The food products are then often transported from the processing facilities to locations where consumers can select and/or consume the food products, such as homes, grocery stores, restaurants, etc.

The processing time for food items, from cultivation until it reaches the end consumer, is generally rather lengthy requiring a number of handoffs before foodstuffs reach the consumer. Such processes primarily include packaging and distribution. A lot of time is typically wasted during those processes, and during transit between the various facilities, generally resulting in a degradation of freshness of food items.

The transit process itself can be challenging as various foods items require, they be kept in specific temperature ranges so that they can be delivered at an optimal temperature to a customer.

### SUMMARY OF THE INVENTION

There is an unmet need for, and it would be highly useful to have, a module for growing and delivering consumable products and/or agricultural products and/or foodstuff, as interchangeably referred to herein as consumable products and/or products. The consumable products and/or foodstuff may for example include hydroponically grown produce or/and cultured foodstuffs such as cultured meats and/or produce and/or fungus and/or bacteria and/or algae any combination thereof or the like.

Embodiments of the present invention is for a system and an apparatus providing a mobile growing module for growing and delivering consumable products and/or foodstuffs. The module and/or mobile body that provides for carrying and housing a system capable of cultivating and nurturing agricultural products or the like consumable products, in particular foodstuff or the like agricultural products and/or cultured products for example including but not limited to produce, cultured meats, cultured proteins, cultured carbohydrates, vegetables, fruits, fungus, bacteria, yeast, algae, fish, shellfish, eggs, seaweed, or the like.

Embodiments of the present invention provide for a system comprising the apparatus in the form of a mobile growing module that may be functionally associated with a vehicle and/or propulsion vehicle wherein the vehicle module may be provided in optional forms.

Embodiments of the present invention provide an apparatus in the form of a mobile module that may be implemented with and/or coupled to optional vehicle modules for example including but not limited to a gravitationally bound and/or earth-bound vehicle for example including but limited to a land-based-vehicle, water-based-vehicle, air-based-vehicle, autonomous vehicle, semi-autonomous vehicle, drone, airplane, manned aircraft, unmanned aircraft, manned ship, unmanned ship, orbiting satellite, or the like or combination thereof.

In embodiments, the apparatus and/or mobile module according to the present invention may be implemented for use in the earth's upper atmosphere and/or external to the earth's atmosphere and therefore configured to be utilized with and/or coupled to a satellite, a space-station, space vehicle, spacecraft, interstellar vehicle, jet propulsion vehicle, rocket, the like or any combination thereof.

In embodiments the apparatus and/or mobile module may feature a coupling interface provided for coupling the mobile module to optional vehicle forms both interstellar and/or gravitationally bound and/or earth bound.

In embodiments the apparatus and/or mobile module provides for rendering such consumable agricultural products readily available in variable environments either earth-bound or in an outer space setting.

In embodiments the apparatus and/or mobile module further provides for allowing consumers to receive and/or purchase the produced goods based on the developmental stages of the product itself. For example, a consumer may select to purchase a 5 day old sprout of one species while selecting a 14 day old sprout of a different agricultural species.

Embodiments of the present invention further provide a method of use of the system that allows a consumer to follow the growth and growing conditions of a particular agricultural products and/or a cultured product so as to meet the consumers need. The consumer may select the product, the age, and accordingly a delivery time will be selected so as to deliver the consumables at the correct developmental stage and selected maturity based on the consumers demand.

A method according to the present invention provides a consumer with a system and device to grow a consumable goods and/or an agricultural product in various environments earth-bound or interstellar. Optionally and preferably the mobile module further provides a user the ability to selected produce characteristics within the system of the present invention while allowing the consumer maximum control so as to allow the consumer to “pick” the agricultural product at the time of his choice based on the selected developmental stage, wherein delivery of the produce is selected according to the developmental stages of the delivered goods.

In embodiments, the apparatus and/or mobile module according to the present invention comprises a framework configured for growing and/or cultivating consumable goods and/or agricultural goods under controlled conditions so as to allow the consumable goods and/or agricultural goods to develop according to controllable environmental parameters, irrespective of the external environment within which is it disposed, earth-bound or outer space.

In embodiments, controlled environmental parameters may for example include but is not limited to at least one or more of: pressure, air composition, water, water flow rate, types of gases, concentration of gases, oxygen levels, nutrient level, ambient temperature, humidity, the like or any combination thereof.

In embodiments the apparatus and/or mobile module may comprise at least two housings including at least one primary housing preferably utilized as a functional growing environment and/or space, and a secondary housing allowing for interfacing with the external environment.

In embodiments the primary housing is preferably an environmentally controlled volume and/or space wherein the environment parameters are controllable and therefore isolated from the external environment.

In embodiments the secondary housing is configured for interfacing between the external environment and the growing environment internal to the primary housing. Most preferably the internal growing environment may be controllable external to the primary housing optionally remotely and/or from a dedicated controlling device associated with the secondary housing.

In some embodiments the mobile module may comprise at least three housings including at least two or more primary housings each preferably utilized as a functional growing environment and/or space, and a secondary housing allowing for interfacing with the external environment, that is shared by the at least two or more primary housings.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic block diagram of an exemplary mobile module and system according to the present invention.

### DESCRIPTION OF THE PREFERRED EMBODIMENTS

The principles and operation of the present invention may be better understood with reference to the drawings and the accompanying description. The following FIGURE reference labels are used throughout the description to refer to similarly functioning components are used throughout the specification hereinbelow.
10 system;
20 auxiliary devices;
100 apparatus/mobile module;
101a primary housing;
101b secondary-interface housing;
102 framework module;
104 cultivation cell housing;
105 environmental control module;
106 pipe network module;
108 conveying module;
110 electronics module;
112 power supply module;
114 memory module;
116 communication module;
118 controller module;
120 sensor module;
122 identification module;
124 location module;
126 display module;
130 housing movement module;
150 coupling interface;
200 vehicle and/or propulsion module;

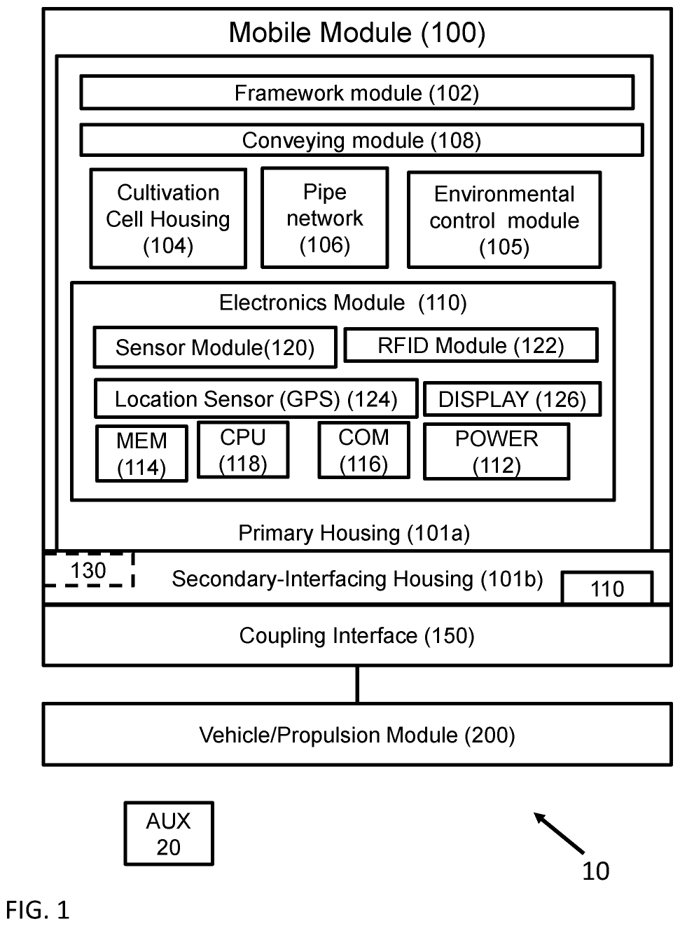

FIG. 1 shows a schematic box diagram of the apparatus according to embodiment of the present invention in the form of a mobile module 100 as used with vehicle and/or propulsion module 200 forming a system 10, according to embodiments of the present invention. Mobile module 100 and system 10 provide for and/or facilitates cultivating and/or growing agricultural products such as plants, fungus, yeast, bacteria, cultured meats, algae, or any combination thereof or the like. In some embodiments the agricultural products may be consumables such as greens, vegetables, cultured meats, cultured proteins, cultured carbohydrates, cultured fat(s) the like or any combination thereof. For example, mobile module 100 may be provided for hydroponic agricultural growth and/or for cultured foodstuff growth. In particular mobile module 100 provides a mobile controlled hospitable environment that is configured for growing and/or culturing and/or cultivating goods such consumable products in variable gravitational environments including earth-bound and/or gravitationally bound environments and/or outer space environments and/or transitional environments and/or gravitationally unbound environments.

In embodiments mobile module 100 preferably comprises an isolated primary housing 101a and a secondary housing 101b.

In some embodiments mobile module 100 may comprise at least three housings comprising at least two or more primary housings 101a each preferably utilized as a functional growing environment and/or space, and a secondary housing 101b allowing for interfacing with the external environment, wherein the secondary housing 101b is shared by the at least two or more primary housings 101a.

Primary housing 101a preferably provides a sealed and/or isolated chamber comprising the functional members utilized to render the mobile module functional so as to provide a favorable and/or hospitable environment allowing for the culturing and growing of a consumable product optionally via hydroponic techniques or the like cultivating and/or culturing techniques as is known in the art and discussed below. Primary housing 101a is preferably a pressure controlled environment allowing mobile module to function under different gravitational environments and/or conditions for example including but not limited to in outer space, the upper atmosphere, on ground, on earth.

In embodiments, the environmental conditions internal to and/or within primary housing 101a may be controlled with an environmental control module 105. In embodiments module 105 may be utilized to control any number of parameters within housing 101a, for example including but not limited to pressure, humidity, temperature, any combination thereof or the like. In embodiments, additional functions of control module 105 with respect to controlling the cultivating and/or growing conditions and/or environment within mobile module 100 is discussed in greater detail below.

In embodiments primary housing 101a may be configured to rotate and/or move a common axis relative to secondary housing 101b. In embodiments, housing 101a may be provided with a housing movement module 130 along its external surface and/or bordering surface corresponding to secondary housing 101b so as to facilitate such optional rotational motion. Optionally housing movement module 130 may be disposed about a surface of the secondary housing 101b. Optionally housing movement module 130, shown in dashed lines, may provide for movement control in any one or more of three dimensional axes. Optionally movement module 130 may be configured in the form of a gyroscope movement module.

In embodiments secondary housing 101b interchangeably referred to as interfacing housing, provides an external housing of mobile module 100 that is capable of interfacing with the external environment. Optionally and preferably secondary housing and primary housing may each feature at least one or more opening that when aligned may provide for a common opening and/or door and/hatch so as to allow movement of consumable goods from internal to primary housing 101a to secondary housing 101b. Such an align-able interface is necessary so as to keep primary housing 101a isolated primary housing 101a from potential harmful external environment and/or environmental conditions, for example that may be experienced in outer space.

In embodiments secondary housing 101b features a delivery window and/or portal configured to receive goods from primary housing 101a and delivers it to a user.

In embodiments secondary housing 101b may optionally and preferably be configured to be in the form of a stationary housing, that may facilitate the motion of primary housing 101a relative thereto.

In embodiments secondary housing 101b may optionally feature at least a portion of electronics module 110 configured to render mobile module 100 functional.

In embodiments electronics module 110 may be disposed partially about both primary housing 101a and secondary housing 101b. Optionally electronics module 110 may be wholly in at least one or both of primary housing 101a, and/or 101b.

In some embodiments electronics module 110 may be disposed external to both primary housing 101a, and/or secondary housing 101b.

In preferred embodiments secondary housing 101b further features a dedicated coupling interfaces 150 that provides for interfacing with the functional requirements and/or members of mobile module 100 internal to primary housing 101a.

In embodiments coupling interface 150 preferably provides critical supply line and waste control line for mobile module 100, for example including but not limited to gas, water, fluids, electricity, power, the like or any combination thereof, so as to render the growing and cultivating functions of mobile module 100 functional.

In embodiments coupling interface 150 further provides an interface for coupling platform and/or securing platform and/or associating mobile module 100 with optional vehicle and/or propulsion modules 200.

In embodiments vehicle and/or propulsion modules 200 may for example include but is not limited to any form of earth bound vehicle or outer space vehicle or the like propulsion means. Optionally vehicles module 200 that may be associated with mobile module 100 may for example include but is not limited to drone, land-based-vehicle, water-based-vehicle, air-based-vehicle, autonomous vehicle, semi-autonomous vehicle, airplane, manned aircraft, unmanned aircraft, manned ship, unmanned ship, orbiting satellite, space-station, space vehicle, spacecraft, interstellar vehicle, jet propulsion vehicle, rocket, the like or any combination thereof. In embodiments any such vehicle 200 may be autonomous, semi-autonomous, non-autonomous, piloted, and/or human controlled.

In embodiments mobile module 100 may be in communication with optional auxiliary communication and processing devices 20, for example including but not limited to smartphones, computers, servers, internet enabled devices, or the like communication and processing device capable of wired and/or wireless communication with mobile module 100.

In some embodiments mobile module 100 may be integrated with and/or incorporated into a transportation device 200.

Mobile module 100 preferably provides a framework 102 comprising a plurality of cultivation cell(s) housings and/or growth cells 104 each for growing and/or culturing a consumable product so as to facilitate hydroponic growth and/or culturing of agricultural goods.

Framework 102 preferably comprises a pipe network 106 for piping a flowing fluid, for example including but not limited to water, culturing medium the like or any combination thereof.

Mobile module 100 is preferably associated with an environmental control module 105 provided for controlling the environmental conditions and/or growing conditions in mobile module 100 and in particular within inner housing 100a, framework 102 and/or vehicle 200.

In embodiments, environmental control module 105 may comprise at least one or more sub-module for example including but not limited to: air filter, water filter, water supply, gas supply, oxygen supply, nutrient supply, temperature control, humidity control, growth control sub-module, pressure control sub-module the like or any combination thereof.

Mobile module 100 may further comprise a conveying module 108 provided for conveying and/or moving products in and around framework 102 and in particular at least one or more cultivating cell(s) housings 104. In embodiments conveying module may for example comprise a conveyer belt and/or the like mechanical structures and/or motors to facilitate mobilization.

Mobile module 100 preferably comprises an electronics and circuitry module 110 provided for rendering mobile module 100 functional. Optionally module 110 may be wholly or partially disposed in any one of or both primary housing 101a and/or secondary housing 101b along any portion thereof.

Electronics circuitry module 110 provides the data management, identification and processing capabilities associated with Mobile module 100 and in particular the system user. Module 110 may comprise one or more of the following: a radio frequency identification module 122, a memory module 114, a communication module 116, a processing module 118, and a power module 112, or the like.

In some embodiments module 110 may optionally further comprise a display 126. Display 126 may be provided in optional form for example including but not limited to a LED indicator, LCD display, LED array, the like or any combination thereof.

In some embodiments module 110 may optionally further comprise a sensor module 120, that may comprise at least one or more sensors. In embodiments sensor module 120 may for example include but is not limited to a temperature sensor, pH sensor, proximity sensor, weight sensor, humidity sensor, oxygen sensor, nutrient sensor, water sensor, camera, light sensor, accelerometer, gyro, flow sensor, optical sensor, infrared sensor, acoustic sensor, image sensor, CO2 sensor, Nitrogen sensor, gas sensor, the like or any combination thereof.

In some embodiments module 110 may optionally further comprise a communication module 116. Communication module 116 may be provided for receiving and/or communicating any data associated with electronics module 110. Optionally communication module 116 may provide for any form of communication as is known in the art for example including but not limited to wired, wireless, contactless, near field communication (NFC), radio frequency communication, optical communication, infrared communication, acoustic communication, Bluetooth, ZigBee, any combination thereof or the like.

In embodiments communication module 116 may be utilized to communication with optional auxiliary devices 20 and/or vehicle module 200.

Processing module 118 may be provided in the form of a central processing unit (CPU) for example in the form of a microprocessor that provides for controlling and processing all data received and/or communicated to or from any portion of electronics module 110.

Memory module 114 may be provided form memory of any form as is known in the art for example including but not limited to flash memory, volatile memory, non-volatile memory, any combination thereof or the like.

In some embodiments data stored with module 114 may be communicated to an external processing device, such as auxiliary device 20, via communication module 116.

In embodiments circuitry module 110 is preferably powered with power module 112. Preferably power module 112 is provided to supply sufficient power to all system associated with mobile module 100 and/or vehicle 200 so as to properly control the vehicle and the environmental conditions required to undertake controlled culturing and/or growing of the agricultural products.

In embodiments circuitry module 110 may optionally include a radio frequency identification chip 122 (RFID), the ID chip 122 is provided to store user identification data, and/or individual parameters preferably associated with individual growth cell housings 104, the data may be communicated over a radio frequency. More preferably, RFID chip 122 is provided in the form of a passive RFID tag, optionally RFID chip 122 may be provided as an active RFID tag.

In embodiments circuitry module 110 may further include a location sensor 124 for example a GPS sensor.

In embodiments mobile module 100 provides for growing an agricultural consumable products, comprising: a structural framework module 102; an environmental control module 105; sensor module 120; controller module 118; and power module 112.

In embodiments the conveying module 108 may comprises a conveyor for mobilizing the consumable products along said structural framework 102. In embodiments, the conveying module may further comprise a weight sensor. In some embodiments conveying module 108 may further comprise and/or provided in the form of a goods movement module. The conveying module may be provided in the form of a robotic arm.

In embodiments the environmental control module may comprise at least one submodule for example including but not limited to: air filter, water filter, water supply, gas supply, oxygen supply, nutrient supply, temperature control, humidity control, growth control sub-module, pressure control sub-module the like or any combination thereof.

In embodiments the sensor module may for example comprise at least one sensor but is not limited to sensors selected from: weight sensor, humidity sensor, temperature sensor, oxygen sensor, nutrient sensor, water sensor, camera, light sensor, accelerometer, gyro, flow sensor, optical sensor, infrared sensor, acoustic sensor, image sensor, the like or any combination thereof.

In embodiments of mobile module 100 wherein the agricultural consumable product is foodstuff for human and/or animal consumption. In some embodiments the agricultural consumable product is an in-vitro cultured product.

In some embodiments the in-vitro cultured product is meat or/and a meat substitute.

In some embodiments the foodstuff is hydroponically grown produce.

In some embodiments the in-vitro cultured product is based on a culturing of an animal cell.

In some embodiments the in-vitro cultured product is based on culturing of a plant cell.

In some embodiments the in-vitro cultured product is based on culturing of fungus.

In some embodiments the in-vitro cultured product is based on culturing of yeast.

In some embodiments the in-vitro cultured product is based on culturing of microorganisms, for example including but not limited to gastrointestinal microbiota.

In some embodiments the in-vitro cultured product is based on culturing of human gastrointestinal microbiota.

In some embodiments the framework module includes a pipe network for conveying a flowing fluid. In some embodiments the pipe network comprises a screw pump.

In some embodiments the framework module may be configured to be portable.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

It should be noted that where reference numerals appear in the claims, such numerals are included solely or the purpose of improving the intelligibility of the claims and are no way limiting on the scope of the claims.

Having described a specific preferred embodiment of the invention with reference to the accompanying drawings, it will be appreciated that the present invention is not limited to that precise embodiment and that various changes and modifications can be effected therein by one of ordinary skill in the art without departing from the scope or spirit of the invention defined by the appended claims.

Further modifications of the invention will also occur to persons skilled in the art and all such are deemed to fall within the spirit and scope of the invention as defined by the appended claims.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

While the invention has been described with respect to a limited number of embodiments, it will be appreciated that many variations, modifications and other applications of the invention may be made.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

While the invention has been described with respect to a limited number of embodiments, it will be appreciated that many variations, modifications and other applications of the invention may be made.

1. A mobile module for growing a consumable product in various environments, the mobile module comprising:
a) at least two primary housing comprising:
i) a structural framework module;
ii) an environmental control module configured to control environmental parameters within said primary housing;
iii) a sensor module;
iv) a controller module; and
b) a secondary housing associated with said at least two primary housing; and
c) a power module; and
d) a coupling interface configured as a supply line interface for critical supply line and waste control line; and
e) a housing movement module that is configured to interface between said at least two primary housing and said secondary housing; that is configured to mobilize said primary housing relative to said secondary housing,
f) a conveying module including a conveyor for mobilizing the consumable products along said structural framework, the conveying module having a weight sensor.

2. (canceled)

3. The mobile module of claim 1 wherein said coupling interface is further configured for providing a coupling platform for associating the mobile module with a vehicle and/or propulsion module.

4. (canceled)

5. (canceled)

6. (canceled)

7. (canceled)

8. The mobile module of claim 1 wherein said housing movement module is disposed along an external surface of said primary housing or said secondary housing.

9. (canceled)

10. The mobile module of claim 1 wherein said housing movement module is provided in the form of a gyroscopic movement module configured to provide movement in any one or more of three dimensional axes.

11. The mobile module of claim 1 wherein said secondary housing (101b) is configured to be stationary relative to said primary housing.

12. The mobile module of claim 1 wherein said primary housing (101a) and said secondary housing (101b) each have an opening that when aligned forms a common opening interface.

13. The mobile module of claim 1 wherein said primary housing and said secondary housing are isolated from one another.

14. (canceled)

15. The mobile module of claim 1 wherein said environmental control module comprises at least one submodule selected from: air filter, water filter, water supply, gas supply, oxygen supply, nutrient supply, temperature control, humidity control, growth control sub-module, pressure control sub-module or any combination thereof.

16. The mobile module of claim 1 wherein the sensor module comprises at least one sensor selected from: pressure sensor, weight sensor, humidity sensor, temperature sensor, oxygen sensor, nutrient sensor, water sensor, camera, light sensor, accelerometer, gyro, flow sensor, optical sensor, infrared sensor, acoustic sensor, image sensor, pH sensor, proximity sensor, CO2 sensor, Nitrogen sensor, gas sensor, or any combination thereof.

17. The mobile module of claim 1 wherein the agricultural consumable product is selected from one of: foodstuff for human and/or animal consumption, an in-vitro cultured product, in-vitro cultured meat, in-vitro cultured protein, in-vitro cultured meat substitute, in-vitro cultured carbohydrate, in-vitro cultured fat, hydroponically grown produce, in-vitro cultured product based on a culturing of an animal cell, in-vitro cultured product based on culturing of a plant cell, in-vitro cultured product is based on culturing of fungus, in-vitro cultured product is based on culturing of yeast, in-vitro cultured product is based on culturing of microorganisms, in-vitro cultured product is based on culturing of gastrointestinal microbiota, in-vitro cultured product is based on culturing of human gastrointestinal microbiota.

18. (canceled)

19. (canceled)

20. (canceled)

21. (canceled)

22. (canceled)

23. (canceled)

24. (canceled)

25. (canceled)

26. (canceled)

27. (canceled)

28. The mobile module of claim 1 wherein said framework module includes a pipe network for conveying a flowing fluid.

29. The mobile module of claim 28 wherein said pipe network comprises a screw pump.

30. The mobile module of claim 1 wherein said framework module (102) is configured to be portable.

31. A system for growing consumable products comprising the module of claim 1 that is associated with vehicle module (200) selected in the form selected from one of: an interstellar vehicle, gravitationally bound vehicle, an earth bound vehicle, a land-based-vehicle, water-based-vehicle, air-based-vehicle, autonomous vehicle, semi-autonomous vehicle, drone, airplane, manned aircraft, unmanned aircraft, manned ship, unmanned ship, orbiting satellite, a spacecraft, satellite, rocket, jet propulsion vehicle, interstellar shuttle, a space-station, a space vehicle, interstellar vehicle, jet propulsion vehicle, a rocket or a combination thereof.

32. (canceled)

33. (canceled)

34. (canceled)
